(19) United States
(12) Patent Application Publication
(10) Pub. No.: US 2023/0072523 A1
(43) Pub. Date: Mar. 9, 2023

(54) PROCESS AND SYSTEM FOR ACELLULAR THERAPY

(71) Applicant: Orgenesis Inc., Germantown, MD (US)

(72) Inventor: Sabina GLOZMAN, Nahariya (IL)

(73) Assignee: Orgenesis Inc., Germantown, MD (US)

(21) Appl. No.: 17/759,647

(22) PCT Filed: Jan. 28, 2021
(86) PCT No.: PCT/US2021/015488

§ 371 Date: Jul. 28, 2022

Related U.S. Application Data
(60) Provisional application No. 62/966,730, filed on Jan. 28, 2020.

(51) Int. Cl. A61K 35/12; A61M 1/02; A61M 1/34

(52) U.S. Cl. CPC A61K 35/12; A61M 1/0281; A61M 1/3486

(57) ABSTRACT

Process and system for acellular therapy in a human subject are provided. The process and system relate to an acellular therapy using therapeutic extracellular vehicles fused to biological material obtained from the subject, via transfusion by extracorporeal systems. The process for acellular therapy is in a subject in need of such therapy.

### CROSS REFERENCE TO RELATED APPLICATIONS

This application claims the benefit of U.S. Ser. No. 62/966,730, filed on Jan. 28, 2020 (and entitled Process and system for acellular therapy), which is incorporated in its entirety herein by reference.

### BACKGROUND OF THE INVENTION

Switch from cellular to acellular cell therapy based on paracrine effect of regenerative medicine is growing exponentially. Both, cell and gene therapy advance towards dual path: allogenic and autologous treatment with and without viral genetic modifications. Over 500 clinical trials are ongoing in the US and similar number in the rest of the world for decades. During the last couple of years, acellular (cell-free) therapy based on use of secretome-derived extracellular vesicles (“EV”s) such as exosomes, exomers, membrane vesicles and mimetics are under intensive growth while in parallel, this field of science is undergoing translation to the clinic. Additional direction of cell free therapy is use of extracellular modified matrix. The regenerative signals secreted by stem cells offer the prospect of acellular therapy. By eliminating cells from regenerative medicine, the therapeutic tools can be stored on the shelf and be readily available for use as “banked” autologous and/or allogenic cell-free therapy. This recent approach based on EV-based tissue regeneration has profoundly changed the field of regenerative medicine by offering promising novel therapeutic options. Development of novel, reproducible and universal technologies for acellular therapy maximizing natural advantages of EVs is therefore still remains long and unmet need.

### SUMMARY OF THE INVENTION

It is a principle object of the present invention to provide a user friendly, reproducible and efficient systems and methods for acellular therapy. The present invention provides intrinsic benefits of EVs, which are relatively more stable than cells to external environment and have natural fusion capacity.

The invention provides a process for acellular therapy in a subject in need of such therapy, the process comprising: a) obtaining a biological material from the subject; b) optionally, eliminating vesical content from the biological material; c) providing a sample of essentially purified therapeutic extracellular vesicles; d) fusing the therapeutic extracellular vesicles of step c with the biological material of step a to obtain a biological material comprising therapeutic extracellular vesicles; and, e) transfusing the biological material of step d to the subject, wherein the transfusion is extracorporeal transfusion.

The invention further provides a system for acellular therapy comprising a)a fusion unit comprising a housing and, optionally, movement-inducing element, said fusion unit is configured to promote fusion of therapeutic extracellular vesicles with a biological material obtained from a human subject to thereby obtain a biological sample comprising said therapeutic extracellular vesicles; b) therapeutic extracellular vesicles supply unit configured to supply a sample of therapeutic extracellular vesicles to the fusion unit; and c)a transfusion unit configured to transfuse the biological material comprising the therapeutic extracellular vesicles to the human subject.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a flowchart illustrating an exemplary embodiment of the process for acellular therapy;

FIG. 2 is a block diagram illustrating an exemplary embodiment of a system for acellular therapy;

FIG. 3 illustrates rEVs measurements by the NanoSight analyzer for quality control;

FIG. 4 illustrates a parallel loop of EV depletion/replacement Wistar rat tail model; and

FIG. 5 is a block diagram illustrating an exemplary embodiment of depletion device.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

Process for acellular therapy

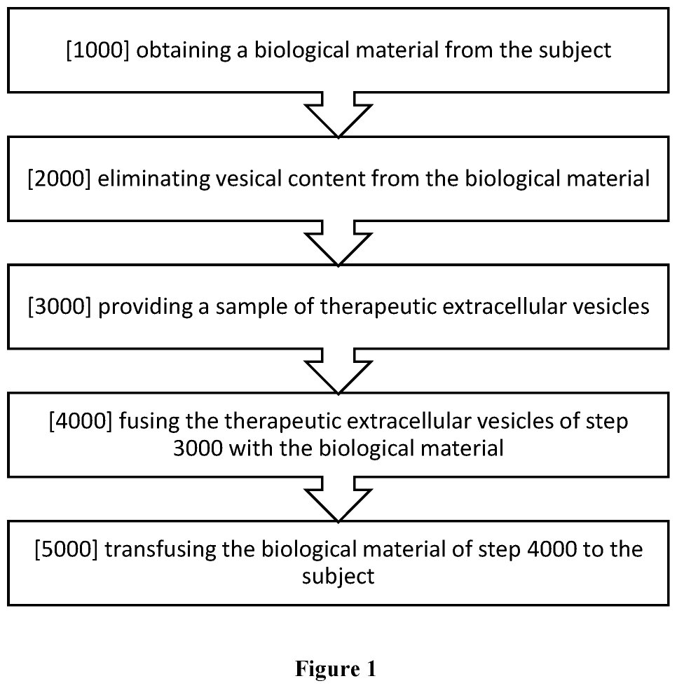

Reference is now made to FIG. 1 illustrating an exemplary embodiment of the process for acellular therapy in a subject in need of such therapy. The process of the invention comprises the steps of obtaining a biological material from the subject [1000]; optionally, eliminating vesical content from the biological material [2000]; providing a sample of essentially pure therapeutic extracellular vesicles [3000]; fusing the therapeutic extracellular vesicles of step c with the biological material to obtain a biological material comprising therapeutic extracellular vesicles [4000]; and, transfusing the biological material of step d to the subject, wherein the transfusion is extracorporeal transfusion [5000].

In the context of the invention the term “Extracellular vesicles (EVs)” is meant to be lipid bilayer-delimited particles that are naturally released from a cell and, unlike a cell, cannot replicate. EVs range in diameter from near the size of the smallest physically possible unilamellar liposome (around 20-30 nanometers) to as large as 10 microns or more, although the vast majority of EVs are smaller than 200 nm. They carry a cargo of proteins, nucleic acids, lipids, metabolites, and even organelles from the parent cell.

In the context of the invention, the term “essentially pure” is meant to be understood as at least partially purified therapeutic extracellular vesicles, namely sample having at least 50% or more fractions of the therapeutic extracellular vesicles. In one embodiment, the sample having at least 60% or more fractions of the therapeutic extracellular vesicles. In another embodiment, the sample having at least 70% or more fractions of the therapeutic extracellular vesicles.

In one embodiment, the non-limiting list of biological material includes: apheresis or hemodialysis accepted vascular system derived fractions, cells, fluids, blood cells fractions, plasma, body fluids, lymphatic system derived cells, adipose stem cells, stromal vascular fraction (SVF) of adipose tissue, bone marrow fractions, including blood cells, red blood cells, platelets , mesenchymal stem cells, hematopoietic stem cells, soft tissue derived cells including fibroblast like cells and mesenchymal cells, synovial fluid fractions from joint aspirates (arthrocentesis), peritoneal dialysis accepted fluids, or a combination thereof. In one embodiment, the biological material is a sample of blood cells. In one embodiment, the blood cells are selected from the group consisting of platelets, red blood cells, lymphocytes, monocytes, peripheral blood cells (PBMS), T cells, B cells, leukocytes, white blood cells, and a combination thereof.

Depletion

According to some embodiments, the biological material collected from a patient suffering from the disease associated with pathological extracellular toxins and pathological endogenous EV risks, may undergo preparation for acellular therapy. In one embodiment, components of the biological material are at least partially purified, from the internal toxic EV content. Selection of disease-associated/invading/toxic factors bearing recipient EVs may be performed by dialysis defiltration-like depletion; may be detected by pathological or specific EVs biomarkers; and/or maybe separated using an affinity column In one embodiment, the purification may be triggered by osmotic, electrolytic, chemical and/or electromagnetic or biophysical triggers.

In one embodiment, the step of eliminating (depleting) vesical content from the biological material can be performed by TFF (tangential flow filtration). In one embodiment, the step of eliminating (depleting) vesical content from the biological material can be performed by size exclusion. In one embodiment, the size exclusion may be performed by purification methods known for viral or exosome particles purification or depletion. Non limiting examples may be Amicon filters, Sepharose based columns (AKTA-GE), cross-filtration/ultrafiltration/tangential flow filtration −TFF devices of Sartorius inc., Pall Inc., Cytosorb (Terumo) or any combination thereof. In another embodiment, the size exclusion may be performed by exosome specific isolation methods and devices. Non limiting examples may be lectin based—Aethlon Medical Inc. (Hemopurifier device (50-150 nm cutoff), IZON size exclusion chromatography; Lonza/HansaBiotech-EV purification from plasma TFF based device (disadvantage-non GMP, small size). In one embodiment, the step of eliminating (depleting) vesical content from the biological material can be performed by affinity binding methods. In one embodiment, the affinity methods use Annexin V, CD31, CD41, CD144, CD146, CD11b, CD61, CD62, CD42, CD66, CD16 or any combination thereof. In one embodiment, the affinity methods use Annexin V. In one embodiment, the affinity methods use CD31. In one embodiment, the affinity methods use CD41. In one embodiment, the affinity methods use CD144. In one embodiment, the affinity methods use CD146. In one embodiment, the affinity methods use CD11b. In one embodiment, the affinity methods use CD61. In one embodiment, the affinity methods use CD62. In one embodiment, the affinity methods use CD42. In one embodiment, the affinity methods use CD66. In one embodiment, the affinity methods use CD16. In the context of the invention, “affinity binding methods” is meant using a filter during EVs depletion that can carry antibodies against specific antigens, and therefore remove any potential pathogenic EVs.

In one embodiment, additional method may be used to obtain maximal purity recovery of EVs from blood for following QC characterization. Non limiting examples may be secondary downstream by size-based centrifugal separation, starch sedimentation, immunomagnetic depletion, lysis followed by a spinning membrane filtration, buoyancy-activated cell separation or any combination thereof.

According to one embodiment, the step of eliminating vesical content from the biological material is performed by dialysis defiltration, affinity column chromatography, immunoadsorbtion, centrifugation, ultra-centrifugation, cross flow filtration, tangential flow filtration, and ultrafiltration. As used herein, the term “eliminating” refers, without limitation to purification, cleaning, elimination of selected, or not selected internal EVs. As used herein, the term “internal EVs” refers to EVs circulating in patient blood that may have a detrimental effect on patient health. A non-limiting example may be, EVs produced by PBMCs (Peripheral blood mononuclear cells) as a result of shear stress experienced during passage in extracorporeal tubing during the hemodialysis procedure, or EVs produced by cancer cells.

According to one embodiment, the eliminated vesicles are collected and characterized as disease diagnostic or disease monitoring parameters. In the context of the invention, the characterization of the vesicles is performed by analytical experiments conducted to detect molecular changes in the protein biomarkers and nucleic acids of isolated EVs, for example, based on antibody-antigen screening by FACS, ELISA, multiplex, antigen-selective beads and other binding assays. In the case of nucleic acid analysis, EVs analyzed post isolation from EV concentrate or pellets by isopropanol or ethanol based purification methods/kits and nucleic acids are analyzed or sequenced for cancer mutations. In one embodiment, monitoring is performed at selected time points during treatment. In one embodiment, the pattern of pathogenic vesicles post replacement sessions should be reduced at least to 20% of the basal level of initial elimination.

Reference is now made to table 1, listing various disease pathologies for identification of biomarkers for disease diagnostic or disease monitoring.

TABLE 1
Quality Control (QC) of EVs based on Identity of Biomarkers of Tumor-derived
EVs:

| | |
|---|---|
| Breast Cancer | BCA-225, hsp70, MARTI, ER, VEGFA, Class III b-tubulin, |
| | HER2/neu (for Her2+BC), GPR30, ErbB4 (JM) isoform, MPR8, |
| | MISIIR, fragments thereof, any combination thereof, or any |
| | combination of antigens that are specific for a breast cancer cell. |
| Ovarian Cancer | CA125, VEGFR2, HER2, MISIIR, VEGFA, CD24 |
| Lung Cancer | CYFRA21-1, TPA-M, TPS, CEA, SCC-Ag, XAGE-lb, HLA Class |
| | 1, TA-MUCI, KRAS, hENTI, kinin Bl receptor, kinin B2 receptor, |
| | TSC403, HTI56, DC-LAMP, hsp70, PIK3CA1391M. |
| Colon Cancer | CEA, MUC2, GPA33, CEACAM5, ENFB1, CCSA-3, CCSA-4, |
| | ADAM10, CD44, NG2, ephrin Bl, plakoglobin, galectin 4, RACK1, |
| | tetraspanin-8, FASL, A33, CEA, EGFR, dipeptidase 1, PTEN, Na(+)- |
| | dependent glucose transporter, UDP-glucuronosyltransferase 1A, |
| | fragments thereof |
| Prostate Cancer | PSA, TMPRSS2, FASLG, TNFSF10, PSMA, NGEP, I1-7RI, CSCR4, |
| | CysLTIR, TRPM8, KvL3, TRPV6, TRPM8, PSGR, MISIIR, |
| | galectin-3, PCA3, TMPRSS2:ERG |
| Brain Cancer | PRMT8, BDNF, EGFR, DPPX, Elk, Densin-180, BAI2, BAI3 |
| Blood Cancer | CD44, CD58, CD31, CDlla, CD49d, GARP, BTS, Raftlin |
| Melanoma | DUSP1, TYRP1, SILV, MLANA, MCAM, CD63, Alix, hsp70, |
| | meosin, pl20 catenin, PGRL, syntaxin binding protein l &2, caveolin |
| Liver Cancer | HBxAg, HBsAg, NLT |
| Cervical Cancer | MCT-1, MCT-2, MCT-4 |
| Endometrial | Alpha V Beta 6 integrin |
| Cancer | |
| Barrett's | p53, MUCI, MUC6 |
| Esophagus | |
| Prostatic | KIAl, intact fibronectin |
| Hyperplasia | |
| Head and Neck | EGFR, EphB4 or Ephrin B2 |
| Cancer | |
| Gastrointestinal | PDGFRA, NHE |
| Stromal Tumor | |
| RenalCell | PDGFRA, VEGF, HIF 1 alpha |
| Carcinoma | |
| Esophageal | CaSR |
| Cancer | |

Biomarkers of EV relevant for CNS and Neurodegenerative diseases:

| | |
|---|---|
| Stroke | S-100, neuron specific enolase, PARK7, NDKA, ApoC-I, ApoC-III, |
| | SAA or AT-III fragment, Lp-PLA2 |
| Prion Disease | PrPSc, 14-3-3 zeta, S-100, AQP4 |
| Parkinson's | PARK2, ceruloplasmin, VDBP, tau, DJ-1 |
| Disease | |
| Alzheimer's | APP695, APP751 or APP770, BACE1, cystatin C, amyloid .beta., T- |
| Disease | tau, complement factor H or alpha-2-macroglobulin |
| Schizophrenia | ATP5B, ATP5H, ATP6V1B, DNM1 |
| Peripheral | OX42, ED9 |
| neuropathic | |
| pain | |
| Chronic | chemokine receptor -CCR2/4 |
| Neuropathic | |
| Pain | |
| Autism | VIP, PACAP, CGRP, NT3 |

QC for EVs of metabolic and cardiovascular disorders:

| | |
|---|---|
| Diabetes | IL-6, CRP, RBP4 |
| Cardiovascular | FATP6 |
| Disease | |

QC of EVs of Infalmmatory and Immune-disorders:

| | |
|---|---|
| Asthma | YKL-40, S-nitrosothiols, SSCA2, PAI, amphiregulin, periostin |
| Rheumatic | Citrulinated fibrin a-chain, CD5 antigen-like fibrinogen fragment D, |
| Disease | CD5 antigen-like fibrinogen fragment B, TNF alpha |
| Multiple | B7, B7-2, CD-95 (fas), Apo-l/Fas |
| Sclerosis | |
| Psoriasis | flt-1, VPF receptors, kdr |
| Autoimmune | Tim-2 |
| Disease | |
| Irritable Bowel | IL-16, IL-lbeta, IL-12, TNF-alpha, interferon-gamma, IL-6, Rantes, |
| Disease (IBD) | 11-12, MCP-1, 5HT |
| or syndrome | |
| (IBS) | |
| Lupus | TNFR |
| Fibromyalgia | neopterin, gpl30 |

Fusion

In one embodiment, the source for the therapeutic extracellular vesicles is selected from the group comprising thrombocytes, platelets, red blood cells, lymphocytes, macrophages, monocytes, or any combination thereof.

In one embodiment, incoming whole blood, or umbilical cord blood (UCB) is analyzed for complete blood cell counts, sterility, and leukocyte viability via flow cytometric analysis using specific antigens on the cell surface of target cell populations including, but not limited to, CD45, CD34 and CD3.

In one embodiment, to induce EV release and protective homeostatic response—donor plasma will be manipulated chemically, physically or combination thereof. In one embodiment, chemically manipulation comprises treatment with ADP, Ach modulators, CaC12, HWHA or combination thereof. In one embodiment, physically manipulation comprises vortex, cold/heat shock, acidic pH=6,2, hypoxia or combination thereof.

In the context of the invention, the phrase “fusing the therapeutic extracellular vesicles with the biological material” is meant to be understood as fusion between the membrane of the EV and the membrane of cellular component of the biological material. In another embodiment of the invention, the therapeutic extracellular vesicles are selected from the group consisting of lipid membrane-associated extracellular vesicles, artificial vesicles, pre-manufactured EV mimetics, exosomes, acellular vesicles, micro-vesicles, membrane vesicles, apoptotic bodies, autophagosomes, cell fragments-derived membrane-containing particles, nano-ghosts, exomers, lipid membrane nanoparticles, extracellular microparticlues, lipid membrane submicron particles, and organelles-containing membrane-associated particles. According to some embodiments, the therapeutic extracellular vesicles are EV mimetics. In one embodiment, EV mimetic is an Excellamer. In one embodiment, Excellamers can be obtained from bio-printed cellular source. In another embodiment, Excellamers may be produced by adopting bio-convergence approach, namely adjusting biopharma particle engineering existing methods and equipment to production of Excellamers. In one embodiment, the methods include, without limitation, 3D organoids and 3D printing technologies; and extrusion-based bioprinting bio-fabrication method. In one embodiment, the source for 3D cultures to source donor EVs are 3D bioprinting techniques to produce organoids, such as cells, tissue, and cell culture. In another embodiment, the approach to prepare cell source raw material for further extrusion, bioprinting of Excellamers™ is roller compactor particle engineering technique for generating micron-sized particles. In one embodiment, the source material may be dried by freeze drying. In one embodiment, the source material nay be dried by continuous sublimation (Lyovapor™ L-300). In another embodiment, the source material may be exposed to aseptic spray drying. A non-limiting example is Buchi 290 Spray Drier that may be upgraded to biological compliancy with aseptic nozzles, thus becoming adapted to GMP standards, that may be further reduced with extrusion technique to form EV mimicking submicron ornano-size vesicles without destroying bio-efficacy. In one embodiment, large scale microspheres may be produced by close loop adaptation of freeze-technologies (Meridion Technologies (Germany) resulting in continuous generation of frozen microparticles. In one embodiment, the particle size of the microparticles is about 250 mcn. According to some embodiments, freeze dry, spray dry, and roller compactor techniques result in micro-sized biological material that may be further downsized by extrusion or and bioprinting technique adaptation. According to some embodiments, 3D-bioprinter (GMPrint™), bio-ink (ColVivo™) may be used to generate regenerative source material to donor EV generation or EV biomimetics. According to some embodiments, extrusion may be performed by Avanti (widely used in state of the art for aseptic and uniform liposome production used formulation nano-size extruder). Additional non-limiting list of systems that may be adopted from the field of particle engineering are micro pellet extruder on a 3D printer and polymeric microsphere extruder (syringe pump), polymers should be changed to biocompatible, e.g. albumin, trehalose, or/and polymers and scaffolds such as Extracellular matrix derived collagens, hyaluronic acid, and fibrinogen. In one embodiment, in order to stabilize and improve quality of EVs and EV mimetics, such Excellamers, freeze-drying of final EV product is a viable alternative to product format in comparison to fresh material. In one embodiment, commercial EVs can be reduced in conditioned medium by TFF (tangential flow filtration) volume concentration with the goal to downstream purification of EVs, while final EV may be freeze dried easily. In one embodiment, lyophilization after repetitive freeze-thawing does not affect the desired EV product stability (by purity and particle size quality control).

According to one embodiment, the particle size of the therapeutic extracellular vesicles is 3000 nm or less. According to another embodiment, the particle size of the therapeutic extracellular vesicles is between 30 nm to 3000 nm. According to one embodiment, the particle size of the therapeutic extracellular vesicles is between 100 nm to 1000 nm. According to some embodiments, the particle size of the therapeutic extracellular vesicles is between 30 nm to 3000 nm; 40 nm to 3000 nm; 50 nm to 3000 nm; 100 nm to 3000 nm; 150 nm to 3000 nm; 200 nm to 3000 nm; 250 nm to 3000 nm; 300 nm to 3000 nm; 350 nm to 3000 nm; 400 nm to 3000 nm; 500 nm to 3000 nm; 750 nm to 3000 nm; 1000 nm to 3000 nm; 30 nm to 2000 nm; 40 nm to 2000 nm; 50 nm to 2000 nm; 100 nm to 2000 nm; 150 nm to 2000 nm; 200 nm to 2000 nm; 250 nm to 2000 nm; 300 nm to 2000 nm; 350 nm to 2000 nm; 400 nm to 2000 nm; 500 nm to 2000 nm; 750 nm to 2000 nm; 1000 nm to 2000 nm; 30 nm to 1000 nm; 40 nm to 1000 nm; 50 nm to 1000 nm; 100 nm to 1000 nm; 150 nm to 1000 nm; 200 nm to 1000 nm; 250 nm to 1000 nm; 300 nm to 1000 nm; 350 nm to 1000 nm; 400 nm to 1000 nm; 500 nm to 1000 nm; and 750 nm to 1000 nm.

Enrichment

According to one embodiment, EVs/exosomes for replacement EV therapy may be analyzed by quantitating the population of exosomes with a particular biomarker profile for purity and identity. In one embodiment, the biomarker may be selected from tetraspanin CD63, tetraspanin CD81, tetraspanin CD9, Alix, flotillin, TSG101, Rab5b, CD82, 14-3-3 proteins, major histocompatibility complex (MHC) molecules and cytosolic proteins such as specific stress proteins (heat shockproteins; HSPs) and the Endosomal Sorting Complex Required for Transport (ESCRT-3) binding protein Alix (46), relevant biopotency functional assay proving protective mechanism of action in selected indication, or any combination thereof.

In some embodiment, the extracellular vesicles are transfused directly into the patient's systemic circulation. In one embodiment, after introduction to systemic circulation the biodistribution of the extracellular vesicles to the patient's organs occurs within up to 24 hours. In another embodiment, after introduction to systemic circulation the biodistribution of the extracellular vesicles to the patient's organs occurs within up to 12 hours. In another embodiment, after introduction to systemic circulation the biodistribution of the extracellular vesicles to the patient's organs occurs within up to 6 hours. In another embodiment, after introduction to systemic circulation the biodistribution of the extracellular vesicles to the patient's organs occurs within up to 3 hours. In another embodiment, after introduction to systemic circulation the biodistribution of the extracellular vesicles to the patient's organs occurs within up to 30 minutes.

In one embodiment, after introduction to systemic circulation, the extracellular vesicles circulate to the liver, the lunges, the kidneys or any combination thereof. In another embodiment, after introduction to systemic circulation, the extracellular vesicles circulate to the liver. In another embodiment, after introduction to systemic circulation, the extracellular vesicles circulate to the lunges. In another embodiment, after introduction to systemic circulation, the extracellular vesicles circulate to the kidneys.

In one embodiment, the extracellular vesicles target inflammation areas.

According to one embodiment, the step of transfusing the biological material of step [4000] to the subject is performed by a recipient-adjusted extracorporeal device. In one embodiment, the recipient-adjusted extracorporeal device is selected from the group consisting of pharmapheresis, hollowfiber dialysis device, standard apheresis cartirage, blood transfusion intravenous device, plasma transfusion device, blood cells transfusion device, cardiopulmonary resuscitation, extracorporeal membrane oxygenation, hemofiltration, cardiopulmonary bypass, arthrocentesis, peritoneal dialysis, intrathecal device, and a syringe pump. According to some embodiments, the architecture design is based on conventional cellular extracorporeal devices. In one embodiment, the conventional extracorporeal devices may be easily adapted to acellular therapy. Some extracorporeal apparatuses and devices are disclosed that are known in the art and are currently in use for cellular therapy and may be adapted to the acellular therapy approach with slight modification in design in order to avoid using cells and using EVs instead. The acellular therapy of the invention provides a game-changing solution by eliminating the risks for failure of extracorporeal cellular therapy that is based on vulnerability of therapeutic cells ex-vivo to recipient circulation, and elimination/depletion of patient cell toxins and pathological disease-associated EVs prior to the EVs fusion. The invention further reduces risks of immuno-and-metabolites/end products—mediated toxicity of donor cells to recipient and also reduces risk of patient cells toxicity—to donor's acellular material.

According to some embodiments of the invention, the step of fusing the therapeutic extracellular vesicles with the biological material is performed, at least in part, in parallel to the transfusion step. In yet another embodiment, the step of fusing the therapeutic extracellular vesicles with the biological material is performed prior to the transfusion step. In one embodiment, the duration of the fusion step is from about 10 minutes to about 12 hours. According to some embodiments, the fusion step is carried out at room temperature (RT) or above. According to another embodiment, the fusion step is carried out at the temperature lower than 37° C. According to some embodiments, the fusion step is carried out in physiologically biocompatible medium and/or buffer. According to some embodiments, the duration of the fusion step is 10 minutes or more. According to another embodiment, the duration of the fusion step is less than 24 hours. According to one embodiment, the fusion step and the transfusion step are carried out in a closed-loop system. In one embodiment, therapeutic extracellular vesicles are prepared at average particle size of 200 nm from known to be regenerative HUVEC (human umbilical vein endothelial cells) derived donor EVs. The fusion is initiated on the 3^{rd} minute of incubation at 37° C. and reaches plateau after 30 minutes meaning that all recipient cells are fused by therapeutic extracellular vesicles. In another embodiment, the recipient cells are completely fused 45 minutes post-initiation at RT. In one embodiment, at RT therapeutic extracellular vesicles are used within 24 hours; preferably within 1-8 hours; most preferably within 30 min-2 hours to avoid co-fusion, aggregation and/or disaggregation and therefore alterations in EVs particle size and integrity. In one embodiment, the fusion is performed at temperature above RT. In one embodiment, the temperature is about 37° C. In one embodiment, the fusion is carried out in suitable medium, at physiological pH and has a duration of between 3 to 45 minutes. In another embodiment, the fusion is carried out for 24 hours or less. In one embodiment, the fusion is carried out for less than 24 hours. In one embodiment, the regular set up of accepted in medical care methods of adjusted apheresis, transfusion or dialysis and other extracorporeal treatment set ups is adapted to the process of the invention. In one embodiment, the biological material comprising therapeutic extracellular vesicles of step [5000] is transfused directly into patient blood by a specialized extracorporeal device. In one embodiment, the device is a mobile pump.

According to some embodiments, the step of fusing the therapeutic extracellular vesicles with the biological material is performed, at least in part, in parallel to the transfusion step. According to some embodiments, the step of fusing the therapeutic extracellular vesicles with the biological material is performed prior to the transfusion step. In one embodiment the fusion is performed prior to blood transfusion using short term fusion of therapeutic EVs in red blood cells from a donor blood bag, in an aseptic closed loop device joined to blood transfusion bag. In one embodiment, the short-term exposure is carried out for a time interval including, without limitation, 15 minutes, 20 minutes, and 30 minutes. According to some embodiments, the fusion step and the transfusion step are carried out in a closed-loop system. According to some embodiments, the sample of therapeutic extracellular vesicles is prepared immediately prior to the fusion step. According to some embodiments, the sample of therapeutic extracellular vesicles is prepared in advance of the fusion step. In one embodiment, the sample of therapeutic extracellular vesicle is cryopreserved. In one embodiment, the sample of therapeutic extracellular vesicles is stored for a desired time interval prior to the fusion step. In the context of the invention, the phrase “desired time interval” is meant to be understood as any time interval, that suits the patient, the care giver, the physician or the procedure involving acellular therapy to be carried out. In one embodiment, the transfusion step immediately follows the fusion step. As used herein, the term “immediately” refers, without limitation, to a time period which does not require any storage of the fused biological material prior to transfusion.

According to some embodiments, the therapeutic extracellular vesicles in the sample carry externally preloaded active therapeutic biomolecules. The non-limiting list of biomolecules includes small molecule, chemical organic moiety, hormone, neurotransmitter, cytokine, prostaglandin, lipid bioactive moiety, oligonucleotide, dipeptide, oligomer, peptide, protein, antibody, vaccine, DNA, viral vector, RNA, antioxidant, phytotherapeutic material, photosensitive material, electromagnetic-sensitive material, quantum dot, oxygen, and NO donor.

According to some embodiments, the biological material comprising the therapeutic extracellular vesicles of step [4000] is stored prior to the transfusion step. In one embodiment, the biological material comprising the therapeutic extracellular vesicles of step [4000] of the process of the invention is cryopreserved.

According to some embodiments, the subject in need is afflicted with a condition that may benefit from acellular therapy. The non-limiting list of conditions that may benefit from acellular therapy includes: metabolic disease, kidney disease, liver disease, diabetes, obesity, NASH, cancer, inflammatory disorder, autoimmune disorder, cardiovascular disorder, post-trauma disorder, orthopedic disorder, burns, wound-related hormonal pathology, genetic disorder, neurological disorder, aging-related condition, post-surgery, post-accident stress-related condition, acute radiation syndrome, infection-related condition, condition related to pharma-toxicity, chronic and acute toxicity, psychiatric disorder, environmental hazards-related toxicity, organ transplantation-related condition, chronic pain, acute pain, neurodegenerative disease, preterm birth-related condition, preeclampsia-related condition, infertility, lung pulmonary respiratory disorder, ischemia, stroke, impotence, skin disorders, condition related to plastic surgery or aesthetic procedures, and blindness.

In one embodiment, the kidney disease comprises chronic kidney disease (CKD). In another embodiment, the kidney disease comprises end stage renal disease (ESRD). In another embodiment, the kidney disease comprises COVID19 related kidney disease.

In one embodiment the condition comprises an aging-related condition. In another embodiment, wherein the condition is an aging-related condition, the acellular therapy comprises plasmapheresis of age-related pathogenic EVs and fusion of young healthy platelet derived donor EVs with patient platelets. In one embodiment, the donor EVs are platelet derived. In one embodiment, the IC derived EVs are isolated from donor blood PBMCs. In one embodiment, the IC derived EVs comprises primary dendritic cells EVs, or cultured T-Cell derived EVs. In one embodiment, the IC EVs impact rejuvenating properties of the patient's stem cells population. In one embodiment, the antiaging pattern improvement is tested by biomarkers comprising p53, Caspase3, PARP1, CD151, Mucin16, CA125, MUC1, CD14, PDL or any combination thereof. In another embodiment rejuvenation properties are tested by testing of hallmarks of ageing, such as enhance migration and recruitment characteristics of stem cells, or prevention of exhaustion of stem cell.

System for Acellular Therapy

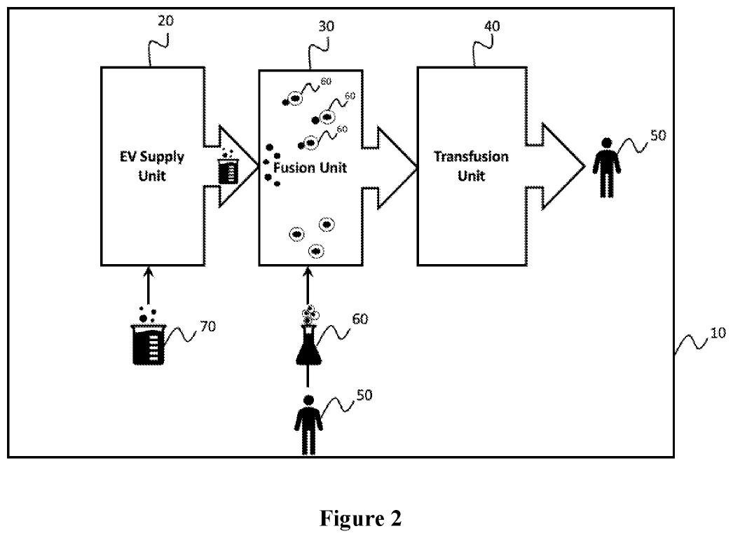

Reference is now made to FIG. 2 providing a schematic representation of an exemplary embodiment of an EXCELLA SYSTEM for acellular therapy. The system for acellular therapy 10 comprises: a fusion unit 30, wherein the fusion unit 30 comprises a housing and, optionally, movement-inducing element, and wherein the fusion unit 30 is configured to promote fusion of therapeutic extracellular vesicles 70 with a biological material 60 obtained from a human subject 50 to thereby obtain a biological sample comprising said therapeutic extracellular vesicles; therapeutic extracellular vesicles supply unit 20 configured to supply a sample of therapeutic extracellular vesicles 70 to the fusion unit 30; and, a transfusion unit 40 configured to transfuse the biological material comprising the therapeutic extracellular vesicles to the human subject 50. In one embodiment, the fusion unit 30 and the transfusion unit 40 are directly connected with each other. In another embodiment, the fusion unit 30 and the transfusion unit 40 are indirectly connected with each other. In yet another embodiment, the fusion unit 30 and the transfusion unit 40 are independent of each other. In one embodiment, the purpose of the therapeutic extracellular vesicles supply unit 20 is for the generation of therapeutic, regenerative EVs from donor sourced cells.

In one embodiment, the EXCELLA SYSTEM is used in addition to an adjunctive extracorporeal device. in one embodiment, the adjunctive extracorporeal device performs as an EV depletion devise. In one embodiment, the EV depletion devise is based on purification or filtration or bioaffinity or size exclusion. In one embodiment, the adjunctive extracorporeal device comprises a dialysis device, an aphersis device, a leukopheresis device or a pulmonary/ECMO-extrocorporeal membrane oxygenation device. In another embodiment, the adjunctive extracorporeal device may be a dialysis device. In another embodiment, the adjunctive extracorporeal device may be an aphersis device. In another embodiment, the adjunctive extracorporeal device may be a leukopheresis device. In another embodiment, the adjunctive extracorporeal device may be a pulmonary/ECMO-extrocorporeal membrane oxygenation device. In one embodiment, the EXCELLA SYSTEM is used in parallel to an adjunctive extracorporeal device. In another embodiment, the EXCELLA SYSTEM is used separately from an adjunctive extracorporeal device.

In one embodiment enrichment system can be connected to existing inlet of extracorporeal machine through disposable cassette including a EV enrichment source inlet for accepting EVs from a fusion unit source. For example, replacement-EV enrichment unit can be added in a closed loop to dialysis systems available for reuse spent dialysate (optionally patient blood or peritoneal dialysate. In such case, the EV replacement unit then will allow really to regenerate the dialysate which is not only depleted form pathogenic EVs but also offer low of fused EVs into dialysate or donor blood cells, fluids (plasma, serum, etc.) as a delivery system for biodistribution of regenerative EVs. The delivery of Evs through said body fluids will be provided by a EV enriched, fused fluid flow tube in fluid connected with the fusion EV source inlet, all in closed loop automated system, optionally with embedded sensors to improve feedback for the automation. The method includes performing continuous flow dialysis with a closed loop hemodialysis with adjunct EV depletion disposable device at a first point in time and performing continuous flow hemodialysis via the same extracorporeal close loop device with adjunct EV replacement post fusion disposable device at a second point in time. In one embodiment, fusion and replacement units may be connected at the same device through close loop aseptic connection inlets.

In one embodiment, the EV depletion unit removes pathogenic EVs components in the dialysate in addition to removal of excess water, toxins, and metabolic wastes. In one embodiment, the regenerative EV replacement unit is added to the dialysate in conjunction with accepted in state of the art additives, e.g. glucose and electrolytes. In one embodiment, for patients suffering from dialysis associated anemia, EV enrichment is added fused into red blood cells for transfusion, optionally at different point of time as accepted in treatment protocol.

### EXAMPLES

### Example 1

### Donor EV Generation from Blood/Platelet-Rich Plasma Fraction-Derived Platelets Neonatal Cell Culture GMP Expansion

Acquiring Blood, Platelet rich plasma (PRP) from activated healthy eligible donors after 12 hours fasting with non-caffeine fluid intake. Young male and female donors (age 18-25) are prescreened for transmitting agents and no chronic or acute diseases. The use of any medication that might affect platelet function should be avoided during 10 days before blood donation. Standardization of cell count in the sample is done by platelet count and determined from this sample with a hematological analyzer. Blood collection to 50 mL is performed in falcon tubes. To obtain platelet rich plasma, 10 mL aliquots of whole blood in 15 mL falcon tubes are centrifugated at 200×g, RT, for 12 min. To the platelet suspension are added (final concentration) 1 mM of MgCl2, 2 mM CaCl2, and 3 mM of KCl. EV stimulators are further added. To stimulate donor-platelet EV release the following agents/treatments are screened: to induce EV release PRP suspension is co-stimulated with 10-20 mcg/mL collagen and 1 U/mL thrombin; 10-20 mcM Ca2+-ionophore; 20-50 mcM of adenosine diphosphate (ADP). As a positive control 1 mcg/mL of lipopolysaccharide (LPS) stimulation is used; as negative control 10% v/v ACD (acid citrate dextrose) and 100 ng/mL Prostaglandin El (PGE1) is added to PRP to inhibit platelet activation.

### Example 2

### EV Supply from Donated Human Plasma by Size-Exclusion Chromatography

10 ml of plasma are loaded on almost dry sepharose column To obtain purified EVs fraction, 35 ml of filtrated fractions are discarded and resulted in EV fraction of —10 mL. To fully dilute high-density lipoproteins, albumin and proteins, about 25 fractions of 5 ml are collected. High-purity and uniformity donor EV supply is achieved by differential (sequential separation) by centrifugation and ultracentrifugation method detailed below. After incubation with stimulators and inhibitors of EV release at 37° C. for 10 to 30 minutes, EVs are separated by centrifugation steps until zero count of cells is found in sequentially separated to new tubes at each step post centrifuged supernatant by following steps:

(1) 5000×g, RT, 5 min following by

(2) 12,000×g, RT, 1 minute (same tube);

(3) 2500×g, RT, 15 min (in a new tube);

(4) 20,000×g, 4° C., for 40 min (new tube);

(5) transfer supernatant into ultracentrifuge tubes: 100,000×g, 4° C., 1-2 h

(6) Donor platelet EV enriched fraction is resuspended with dilution of the pellet to PBS and ultra-centrifugated at 100,000×g, 4° C., 1-2 h and discard the supernatant and the use obtained EVs fresh or stored at Revco, or liquid nitrogen. Donor derived EV platelets are thawed immediately before Fusion step by incubation 2-5 minutes in transfusion buffer before exposure to the recipient blood cells that undergone EV depletion in order to purify disease-associated recipient EV to donor regenerative EVs.

### Example 3

### Effect of Method of Isolation of Donor EVs and Time of Exposure on Fusion Dynamic and Stability of EVs Measured as Particle Size and Purity of Stimulated Neonatal Selected Donor Source Cells

To calibrate the EV-induction of pre-stimulation effect, donor source cells were labeled by 20 mcM of BioDiPY TR fluorecent Texas Red (ThermoFisher, used marker was diluted in DMSO) BioDipy was incubated with pre-stimulated donor EV source cells for overnight in 16 cm petri dish grown selected primary cells sourced from ATCC cell culture collection, in medium suggested by supplier. Once full uptake Calcium ionophore stimulation/or full FBS, growth factors and glucose source starvation was triggered for 24 hours. EV induction was performed with 50 mcM of A23187 (Sigma) for overnight, at the latter case, the FBS (vesicles depleted—ThermoFisher) supplemented medium was replaced with phenol free DMEM with GlutaMax (Invitrogen-ThermoFisher). PhenolRed-free conditioned medium aliquots were measured by fluorimeter (Shimadzu) to calibrate uptake and release of BioDipy fluorescent ceramide incorporation into cell membranes and exocytosis into medium during vesicular stimulation under stress. Once EV induction conditions were calibrated, EV isolation experiment was started.

Selected human-derived donor cells for research use were expanded to 100 million cells per 175 cm² flask per each experiment. Cells were grown in medium recommended by ATCC. 24 hours before stimulation and 24 hours during EV induction/stimulation-FBS containing medium was exchanged to serum free medium and medium was exchanged to Phenol Red free Hanks Buffer Salt Solution plus Hepes for designed time. Quality Control—QC was performed by particle size measurement by Malvern protocol or NanoSight instrumentation (purity >80% of vesicles at size 100-300 nm, the accepted QC specification was 70% of EVs with diameter of <500 nm). EV potency assay for regeneration potential was characterized by scratch assay (CytoSelect kit, Cell Biolabs Inc.) vs platelet activation inhibited negative control and blank. Regenerative index of LPS stimulated platelets' EVs was accepted as 100%, vesicle control showed 20% closure of scratch assay. Negative ACD/PGE1 pre-treated platelet derived EVs shown 30% and A25184 stimulated and thrombin/collagen pre-stimulated platelets derived EVs demonstrated 65 and 70% regenerative (measured as scratch closure rate index) potential. From selected experiments, donor EVs were characterized by particle size, purity, stability in 24 hrs. For selected EVs we demonstrate the correlated rate of fusion (in BiodiPY experiment of relevant examples are shown in arbitrary units, as percentage of basal level) demonstrated in the Table 2.

TABLE 2

| | | | |
|---|---|---|---|
| Type of donor EV, | Particle | Fusion at 10-30 min at | Stability |
| Method of isolation | mean | 25oC | 24 hrs |
| | size, | | |
| | Purity | | |
| HUVEC | 156 nm, | 15-70% | Aggregation: |
| Stimulated (A23184 | >85% | | Particles ize |
| calcium | | | |
| ionophore) | | | changed: |
| Differential | | | >90% |
| centrifugation- | | | |
| ultracentrifugation | | | >1000 nm |
| downstream | | | |
| protocol | | | |
| Self-donated PRP | 560 nm, | 25-60% | >80% |
| platelet rich | >70% | | 1-3400 |
| plasma | | | |
| TFF concentration | | | |
| hollowfiber cutoff | | | |
| 500 kDa, | | | |
| Placental cells | 100 nm, | 30-80% | nd |
| isolated | | | |
| Excellamers by | >90% | | |
| Avanti-extruder | | | |
| aseptic technique | | | |

### Example 4

### EV Depletion and Isolation Methods

Main impurities are blood derived albumin and immunoglobulin that may hinder isolation of EVs from blood of the patient. In order reduce albumin impurity, ultrafiltration by the TFF (tangential flow filtration) method is the most practical and can be performed in a close loop GMP (Good Manufacturing Practice) compliant system. EVs may be concentrated or isolated by size exclusion chromatography or anion exchange and/or gel permeation chromatography as described in U.S. Pat. No. 6,899,863 (incorporated herein by reference), and sucrose density gradients. Alternatively, affinity binding methods can be used, for example, using antibody conjugated magnetic beads methods (for example, from Miletenyi Biotec Inc., or BioSep Ltd.). Purification may be based on CD63 tetraspanin antibody or pathological marker such as EpCam or HSP70, or other selected biomarkers. The presence of high impurity levels in EV preparation used to generated affinity matrices might result in lower efficiency and lower binding specificity when used in the context of apheresis.

In the current specific example, EVs are purified from blood using specific depletion methodologies that deplete EVs larger than 50 nanometers nm or more up to 500 nm in size. Such methodology allows retention of human serum albumin and immunoglobulins—IGs in depleted blood. In order to retain immunoglobulins and HAS-human serum albumin in depleted blood, depletion device pore size should be higher than maximal length, diameter size of HSA/IGs: selected minimal size parameters—more than 70 kDa per molecular size; more than 15 nm per diameter and more than 115 A—angstrom size. As an upper limit to be retained in the blood we select lowest thickness point of blood cells ˜0.8 mcn. Therefore, the selected filtration/size exclusion limit of the depletion device is in the range between 20 nm to 750 nm, preferably 30-700 nm, most preferably 50-500 nm. Size exclusion chromatography is the preferred method to deplete or exclude pathologic patient EVs and retain in circulation the remainder of the blood components.

In order to characterize EVs from depleted permeate, an additional step of ultracentrifugation is possible. Purity qualitive specification of size exclusion should be 70% of EVs in permeate and post ultracentrifugation for biomarkers identification—>80%, preferably >85%, most preferably ˜90% and more.

Depleted EVs are optionally collected as disease diagnostic/monitoring parameter. Monitoring is performed at selected time points during EV replacement associated treatment. Pattern of pathogenic EVs post replacement sessions should be changed at least to 20% of basal level of initial depletion.

### Example 5

### Platelet and CM MSC EVs Protocols of Isolation and Experimental Design

EVs Depletion

During dialysis process, patients undergo pathogenic EVs depletion, which bears the phenotyping markers of shared/specific platelets derived markers.

Depletion is generated either by size filtration, specific antibody binding filter, ion exchanger or a combination thereof.

The instrument is designed to be easily implemented upon any dialysis instrument, by directly intersecting with its outflowing tubing and preserving total cell populations to flow back into the patient. Remaining, smaller in size fractions, will enter a second chamber, which then undergo filtration to preserve pathogenic EVs from re-entering the patient's veins.

The filtration system includes pressure sensors at both instrument's ends, to keep general dialysis pressure intact, should any of the intersections/filters increase pressure flow.

EVs Replacement

Therapeutic EVs are aimed to be transduced during dialysis, only once the pathogenic EVs depletion has been complete.

Allogeneic platelets EVs are generated in a patient independent manner.

Unmanipulated, induced platelets are incubated to secrete EVs into their growth media and preserved in −80° C. until isolated.

EVs are isolated under Tandem Flow Filtration (TFF) system, to preserve size fraction of 50-500 kDa particles.

During TFF, EVs are formulated, and concentrated. Concentrated fractions are sent for analysis and aliquots are preserved at −80° C. At a later stage, a lyophilization process is taking place, to ensure maximum preservation stability.

Generated EVs are categorized under their blood type and stored in an EVs bank reservoir.

For achieving best efficiency of EV transfer to patients, EVs are further categorized into HLA (human leukocyte antigen) sub-types at a later stage to prevent unwanted immune rejection of EVs or exacerbated inflammation in these patients.

For infusion into patient blood, or serum, thawed aliquots are reconstituted with sterile isotonic solution, and infused into the patient as the last treatment step during dialysis.

### Example 6

### Dialysis Collected EVs and Relevant Biomarkers of Extracorporeal EVs Review. QC Markers for Characterization of Depletion d-EVs from Hemodialysis Patients

To quantify the removal of pathogenic EVs as described above, EVs are collected from patients that have completed a hemodialysis treatment (either with or without the EV filtration and removal process) and quantified for expression for EV markers and total EV numbers. The filtration and separation process are designed to reduce the number of EVs, particularly those emanating from platelets.

Markers for Disease Associated EVs in Chronic Kidney Disease:

Chronic renal failure is accompanied by endothelial activation and a large increase in microparticle numbers with reduced procoagulant capacity. The primary marker is Annexin V for detecting phosphatidylserine, which has previously revealed an overall increase in table 1EV levels in disease patients.

Disease associated EVs derive mainly from platelets (95% of all the EVs derive from platelets) and co-express CD41 (common to all platelets) as well as CD63 (8-fold higher levels in disease EVs compared to healthy controls), as well as CD62, CD45, CD34 and CD3.

Methods:

Blood samples are drawn with a 21-gauge needle after applying a light tourniquet. After discarding the first 4 mL, blood is collected into a 4.5-mL tube containing 3.2% trisodium citrate [Becton Dickinson (BD), Plymouth, UK]. Plasma is prepared within 20 min after blood collection by centrifugation for 20 min at room temperature at 1550 g, without a brake. Aliquots of plasma are snap frozen in liquid nitrogen and then stored at −80° C. until use.

EV Separation

In order to isolate the microparticles (MPs), 250 μL of plasma are thawed on ice for 60 min and then centrifuged for 30 min at 17,570×g at 20° C. Subsequently, 225 μL of supernatant (i.e. microparticle free plasma) are removed. The remaining 25 μL containing the microparticle pellet are resuspended in 225 μL of phosphate-buffered saline (PBS; 154 mM NaCl, 1.4 mM phosphate, pH 7.4), containing 10.9 mM trisodium citrate to prevent coagulation activation. Samples are centrifuged for 30 min at 17,570×g at 20° C.; thereafter, 225 μL of supernatant are removed and the microparticle pellet is resuspended in 125 μL of PBS.

For flow cytometric analysis (from Trappenberg et al 2012), MPs (5 μL) are diluted in 35 μL phosphate-buffered saline (PBS) containing 2.5 mmol/L CaCl 2 (pH 7.4). Then, 5 μL Annexin V-APC from Caltag Laboratories (Burlingame, Calif.) and 5μL fluoresceinisotyocianate (FITC), phycoerythrin (PE) and/or peridinin chlorophyll protein complex (PerCP)-labelled cell-specific monoclonal antibodies (mAb) or isotype-matched control antibody are added. CD63-FITC (H5C6, IgG 1) and CD41-FITC (5B12, IgG 1) from DAKO (Glostrup, Denmark) are used, and CD62P-PE (CLB-Thromb/6, IgG 1) from Immunotech (Marseille, France); CD62E-FITC (HAE-1f, IgG 1) from Kordia (Leiden, The Netherlands). Labelled isotype controls IgG 1 (X40) and IgG 2a (X39) are from BD (San Jose) and IgG 2b-PE (MCGb) from IQProducts (Groningen, The Netherlands).

The mixtures are incubated in the dark for 30 min at room temperature. Subsequently, 760 μL

PBS/calcium buffer are added. All samples are analyzed for 1 min during which the flow cytometer analyzed —55 μL of the suspension. The samples are analyzed in a FACS Calibur flow cytometer with CellQuest-pro software (BD). Forward scatter (FSC) and sideward scatter (SSC) are set at logarithmic gain. To distinguish MPs from events due to noise, MPs are identified on FSC, SSC and annexinV positivity. To identify annexin V-positive events, a threshold is placed in a MPs sample prepared without calcium. They are further categorized by binding of a mAb directed against a cell-specific antigen. To identify MPs that bound cell-specific mAbs, MPs are incubated with identical concentrations of isotype-matched control antibodies to set the threshold. Some antibodies have higher background fluorescence than the isotype-matched control and with these antibodies, the threshold is set on the population MPs negative for the antibody.

### Example 7

### Functional Assays

Functional assays are employed as necessary during characterization to ensure that sufficient rEV per dose is present and bears the expected functional characteristics.

Depending on proposed mechanism of action, a potency test is selected in order to release a batch of replacement/regenerative EVs (referred to as rEVs) for fusion into native pathogenic EV depleted blood cells. An assay for measuring the regenerative potential of EVs can be selected from a battery of relevant potency assays that are suited for the study of the target mechanism of action. For example, the scratch assay is selected for measuring regenerative potential of rEVs. Primary human foreskin keratinocytes (HFK) (up to 5th passage) treated with exosomes derived from Adipose MSCs, EVs derived of placental cells (ZenBio), or amnionic fluids derived in house. For treatment normalized EV particles concentration of billion particles/ml, that is approximately equivalent to ˜150 μg/mL of protein is used for treatment. EVs are purified with SEC isolation (0.2 or 0.4 mcn size exclusion cut off filtration) resulted in >90% of purity. Purity is measured by HSA (human serum albumin) quantification as a partial quantity of total protein—reduced ˜100 times post SEC treatment. As a read out of the assay, the percent gap closure measured from the basal scratch width 24 hours post treatment. Positive control is treated with bFGF at 20 mg/ml. Negative control—untreated scratch well. Untreated negative control resulted in >20% of scratch closure, all tested EVs resulted in >80% closure of the scratch width. Therefore >70% of functional potency is selected as batch release qualification for clinical batch. The scratch assay is performed as a biopotency regeneration rEVs testing method, by creating a “scratch” in a cell monolayer using a 10 μl tip. Regenerative effect is evaluated by cell migration, observed by imaging of the scratch area in time intervals and measuring the scratch area length. Cells were initially seeded on a 6-well plate in complete DMEM low glucose medium supplemented with 10% fetal bovine serum (FBS). As FBS components have a positive effect on cell wound healing ability, FBS percentage was gradually reduced from 10% to 1.5% during the 24 h prior to the experiment and then substituted with HBSS-HEPES condition medium for 2 h.

### Example 8

### Using the Donor rEVs Isolated from Healthy Donor Platelets Ex Vivo for Use in Manufacture of a Product for Transfer into a Dialysis or Apheresis or EKMO Patient

The extracorporeal system may be coupled to one or more of the needle, tubing, and receptacle. Apheresis of blood from donors involves separation of blood into different constituents, isolating one or more constituents, and returning the remainder to circulation. For example, leukapheresis entails the isolation of white blood cells from the blood of a donor and returning the remaining cells and plasma to the donor's body. Apheresis methods and devices are known in the art and described in, for example, U.S. Pat. No. 9,364,600; and U.S. Pat. No. 6,743,192 (both incorporated herein by reference). Donor rEVs are prepared in blood banks, GMP manufacturing site centralized or most preferably Point of Care unit. Donor blood or fraction thereof, such as platelets units, is required to arrive at the manufacturing site with minimal hemagglutination, red cell lysis, and cell clumping, and with high leukocyte viability to facilitate downstream processing of red blood cell and platelet depletion. Whole blood is collected in devices that allow blood to be maintained at a controlled temperature and shipped in a stable sterile transport container. The collection devices are also compatible with closed system bioprocessing compatible with GMP.

Blood is stored in containers composed of a sheet material having a plurality of layers where a first sheet which contacts the blood substantially prevents the activation and adhesion of blood platelets to the layer.

rEV Intermediates and Final Drug Product Formulation

As per stability data—vials can be stored in −20° C. prior to administration. The resulting donor platelets derived rEV isolated during the manufacturing process are combined at a ratio of 1:2 with saline/40% dextrose for parental solution to generate the drug substance—each dose is dispensed into 3-4 final drug product containers just prior to cryopreservation and then lyophilized for storage. Post thawing each dose is dispensed into the fusion unit and incubated 5-20 min at RT or 37° C. to accelerate fusion with post internal EV depleted patient/or blood cells EV replacement process pre-formulated donor rEVs and patient EV depleted blood cells are combined by fusion to generate the rEV enriched patient blood final product. Optionally, adjuvant agents can be added during the EV replacement process, such as anti-inflammatory agents, steroids, mTOR inhibitors, stabilizers etc. For a clinical regimen prior to administration human serum albumin (HSA) and Hyaluronic acid (HA) (IVF stabilizing sterile solution—can be purchased ready to use from IVF reagents suppliers), can be added to the EV formation, preferably containing ˜2% of HSA. Labeling of vials (volume of 100-500 mcL) includes batch number, data of production, expiry date (one year shelf life as currently tested in the in the stability protocol) and protein concentration (batch vials should contain 100-500 mcg protein in 100 or 500 mcl, respectively, and optionally, only particles less than one micron will be accepted as per quality control specification: 1E-3E).

### Example 9

### EV Replacement Regimen

The purpose of administration and/or Serial Administration of replacement/regenerative EVs (rEVs) is therapeutic benefit. EV plasmapheresis, preferably as a method to delay progression of hallmarks of aging based on personalized rate of hallmarks progression per patient basal level before the treatment with rEVs, can reduce pathologic EVs prior administration of a regenerative/therapeutically functional rEVs enabling efficient fusion of upon re-placement. This process can be performed in a serial manner over an extended period of time to gradually increase the level/dose of EV replacement in human subjects.

Alternatively, Bioxomes can be used for plasmapheresis as EV “empty” mimetics, which are membrane derived particles that lack a proteins and nucleic acids.

### Example 10

### Depleted EV Characterization

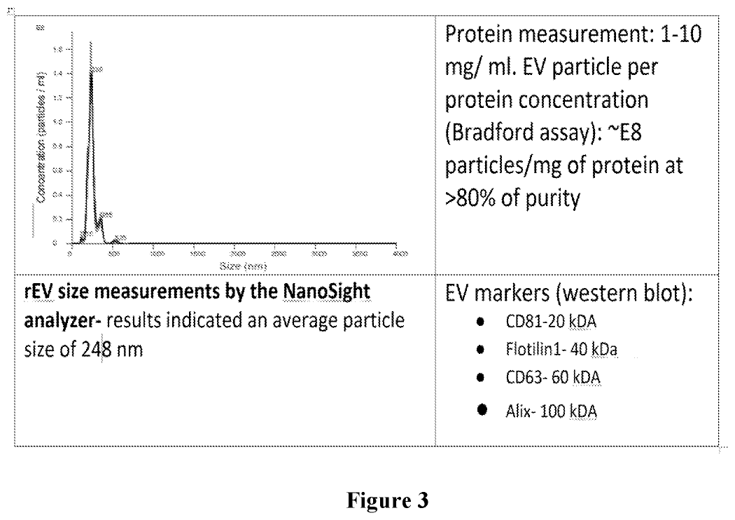

5 mL sample of whole blood is mixed 1:1 with prefixation buffer and incubated for 15 minutes. A biocompatible polymer-based filter from Polysulfonate or silicone with pore size of diameter average ˜50-500 nm is placed into a filter holder and washed with 5 ml PBS. In current example, 500 kDA cut off was selected, equivalent approximately to avg size of EVs of 150-250 nm (see representative QC data on FIG. 3).

Blood is filtered through the filter over 3 minutes and the filter is washed with 10 mL PBS. The EVs on the depletion filter are post-fixed followed by washing. An antibody solution is added and incubated for 1 hour. Resultant EVs biomarkers are compared between rEV treatment time points and analyzed in correlation with clinical parameters. Filtered samples from six patients subjected to EV isolation are analyzed for pathological markers. Blood is taken at 2 time points post replacement (3-4 week apart) in three patients, and the EV results biomarkers pattern is compared. The biomarker differences being found in the same patient samples at both time points correlates with clinical parameters and relevant characterization of pathological biomarkers tests.

### Example 11

### Anti-Ageing EV Replacement

To test EV antiaging pattern improvement, the increase in the ratio between post-EVs replacement and initial basal patient level of at least one of the following platelet EVs biomarkers is examined p53+Caspase 3+PARP1)/(CD151+Mucin16/CA125+ MUC1+CD14+PDL (in a sum or separately), most preferably the p53/CD151 ratio may increase after at least two or three sessions of EV replacement within a three year period. An alternative method for assessing clinical efficacy of anti-aging EV replacement therapy is testing the effect of patient “aged” internal ECs on donor PBMCs, in a PBMC aging assay improvement in patient EV function or markers may be observed over the course of several years of EV replacement therapy. Notable increase of EV concentration offer a possibility to divide between EV depletion and EV replacement regimens in time. Depletion of plasma EVs should be performed at least once annually, but rEV replacement may be performed each quarter by rEV intramuscular injection. Subsequently the ratio of optional biomarkers, e.g. platelet EVs p53/CD151, is tested annually for three years at least to monitor the effect on EV status as a selected intercellular communication ageing hallmark and clinical read out in placebo controlled clinical study. Optional regimen scheme is described below—

1. Deplete recipient/patient ageing/disease-induced pathogenic EVs from patient plasma by magnetic beads or filtration (cut off 500-1000 kDa);

2. GMP (Good Manufacturing Practice) supply of lyophilized batch donor rEV (isolated from activated platelets—functionally characterized) or perinatal rEVs exposed to sub-stress conditions such as heat, hypoxia, chemical, mechanical stress, hyperbaric stress;

3. EV Replacement: Fuse rEVs with donor post-plasmapheresis plasma during 15 min at 37° C. in close loop fusion bag and/or transfuse into patient, optionally by intramuscular route at least once. Post therapy treatment measure platelet EV p53/CD151 ratio or internalization rate of patient monocytes compared to basal levels of activity or marker expression in pre-treatment patient platelets or cells, respectively (or similar cells taken from a and control placebo group).

Various animal models are used to test age-related changes and to select protective anti-ageing methods and compounds of treatment. Examples may be normally aged model animals (e.g rats at over age 22 weeks) models, transgenic mice models that mimic ageing (e.g p16-3MR transgenic mice as described in U.S. application Ser. No. 15/067,543(incorporated herein by reference)) and aged genetically stabilized mice models, e.g. C57BL/6J males and females between 25-90 weeks of age as described in by U.S. Pat. No. 8,110,721(incorporated herein by reference). As for testing the effect of EV depletion replacement treatment in aging, a variety of animal models is described and can be adopted from Alicia R. Folgueras, Sandra Freitas-Rodríguez, Gloria Velasco, and Carlos López-Otín 2018. Mouse Models to Disentangle the Hallmarks of Human Aging https://doi.org/10.1161/CIRCRESAHA.118.312204Circulation Research. 2018; 123:905-924 (incorporated herein by reference).

The present invention suggests to use donor matched or banked PBMC derived EVs to regenerate aged patients' blood that is post leukopheresis, where age-damaged immune cells are eliminated and replaced by EVs of immune cells form donor matched or patient own (banked at younger age). Immune cell EVs known to rejuvenate stem cells pool, improve migration and delay exhaustion of stem cells which is important hallmark of ageing.

### Example 12

### Fusion of Therapeutic Extracellular Vesicles with Red Blood Cells from a Standard Blood Transfusion Bag, Prior to or During Transfusion of the Therapeutic EV-Fused Red Blood Cells into Hemopheresis Patients

Kidney disease patients often suffer from anemia and require transfusion of red blood cells from blood donors during the hemodialysis treatment session. Delivery of therapeutic extracellular vesicles to the patient is carried out in two stages 1) by fusing the EVs to donor blood cells and 2) transfusing the donor blood cells into the patient. This process is mimicked in vitro, as follows. A blood transfusion bag containing donor red blood cells (300 ml) is attached by tubing via a peristaltic pump to a Y-infusion connector, which is connected to a 1-liter flask placed on a rotating table shaker in a 37° C. incubator mixing chamber. A syringe containing 30 ml of therapeutic EVs resuspended in saline and equilibrated at room temperature is attached to the second input of the Y-infusion connector by tubing, via a peristaltic pump. The rate of donor blood flow into the Y-infusion connector is set at 10 ml per minute. The rate of therapeutic EV medium flow into the Y-infusion connector is set at 1 ml per minute. The period of mixing of EVs with donor blood is set to 45 minutes from the initiation of flow into the fusion unit. The mixed EV/donor blood is then analyzed for particle size using a Nano-sight analyzer, to determine the proportion of EVs not incorporated into the red blood cells: a 10 ml sample of the red blood cell/EV mixture is centrifuged at 1000 rpm for 10 minutes to pellet red blood cells. The cell pellet is resuspended in PBS and incubated with anti-CD41 antibodies conjugated to FITC. Red blood cells expressing CD41 are identified using flow cytometry.

### Example 13

### Use of Excellasystem for Regeneration Post Acetominophen Induced Cytotoxicity

Excellasystem can be used in treatment of chronic and acute toxicity. For example, acetaminophen-induced hepatotoxicity has been observed worldwide and accounts for alerting numbers of overdose-related acute liver failure and liver transplant cases. The evidence for extracorporeal treatments in cytotoxic poisoning was recently described (Harbord N. Common Toxidromes and the Role of Extracorporeal Detoxification. Adv Chronic Kidney Dis. 2020 January; 27(1):11-17. doi: 10.1053/j.ackd.2019.08.016. PMID: 32146996. (incorporated herein by reference)).

In the current example, cytotoxicity-induced paracrine EVs were replaced by current existing extracorporeal regimen, in addition to replace depleted body fluids with protective EVs fused during hemodialysis. EV mimetics—Bioxomes or chelator-containing EV mimetics, named Redoxomes as described by us recently in PCT publication no. PCT/IL2019/050391 (incorporated herein by reference) were used. Selected chelator, DSFX—deferoxamine embedded in Redoxomes can be fused with EVs or body fluids, or cells to protect from cytotoxicity damage, prevent liver and kidney failure and mortality, morbidity rate. To establish growth conditions sensitive to proliferation inhibition cytoparameters, calibration of dynamic cell growth parameters was performed by calibration of cell number in CellTiter-Glo Assay (Promega) shown on tables 3 and 4 below in relative units.

TABLE 3

| | AVG RLU | CV |
|---|---|---|
| RCB001 (1000 cells) | 54190 | 7% |
| 10% serum | | |
| RCB001 (5000 cells) | 257500 | 16% |
| 10% serum | | |
| RCB001 (10000 cells) | 506000 | 12% |
| 10% serum | | |
| RCB001 (20000 cells) | 755750 | 3% |
| 10% serum | | |
| Hep2G (1000 cells) | 39104 | 5% |
| 10% serum | | |
| Hep2G (5000 cells) | 185500 | 6% |
| 10% serum | | |
| Hep2G (10000 cells) | 329000 | 6% |
| 10% serum | | |
| Hep2G (20000 cells) | 565500 | 3% |
| 10% serum | | |

Results—Improved viability post Bioxome fusion on cytotoxic model was established and presented in the table below.

Specific protective anti-cytotoxicity example tested on calibrated above assays of EV mimetic proprietary Bioxome, is shown in table below. Cell viability, %, measured by XTT assay (Biological Industries Beit Haemek). Cell viability without treatment was calibrated by dose dependence to serum conditions and cell density. Cell seeding established for 10000, 20000 cells/well in 96 well plates. For RCB001 cells (liver derived mesenchymal like cells)—10,000 cells. For −HepG2 cells—20,000 cells density used. Cells were grown in DMEM plus 10% exosome depleted FBS (ThermoFisher Scientific).

TABLE 4

| | | |
|---|---|---|
| Liver cell type, | Doses tested dependency. | EV mimetic |
| 48 hrs incubation | (2 experiments, n = 3 at each) | fusion effect |
| post-treatment | Control (no treatment)—100% | |
| | viability | |
| RGB- | 5 mM—57-81% viability | Bioxome treatment at 5 |
| Acetaminophen | | mM: 81-96% |
| | 10 mN—46% | |
| HEPG2 | 5 mM—62% | Bioxome treatment at |
| Acetaminophen- | 10 mM—46% | 10 mM: 68-81% in |

### Example 14

### Biodistribution of Fluorescently Labeled Exosomes Following Intravenous Administration to Balb/C Mice

In the current study, the biodistribution of Exosomes coupled to Cy7 fluorophore following IV administration was examined Mice are commonly used species in accordance with international recommendations and the published literature. The Balb/C strain used in this example is a well-known laboratory model with sufficient historical data.

Materials:

Bioxome™ Nanoparticulate were manufactured by Orgenesis. The fluorophore are Cy7, SE, manufactured by TOCRIS. The vesicle is saline. Test items were marked “Vehicle”, “Low Dose” or “High Dose” according to contents. Mice Balb/C were purchased from Envigo RMS (Israel) Ltd.

Study Design and Time Line

The study was conducted in one cycle. The test item was administered IV into the tail vein at dose volume of 200 μL/mouse on Day 1. The test item was administered at three dose levels according to the group design in Table 1. A total of three animals per group were used in Groups 4M and 5M and one animal per group in groups 1M-3M. The experimental design and time line are presented in Table 5 and Table 6, respectively.

TABLE 5
Group Allocation

| Group | No. of | | Route of | Termination |
|---|---|---|---|---|
| No. | Animals | Test item | administration | time |
| 1M | 1 | Vehicle (auto-fluorescence) | IV | 24 hours |
| 2M | 1 | Cy7 (low dose) | (0.2 mL/animal) | post-dosing |
| 3M | 1 | Cy7 (high dose) | | |
| 4M | 3 | Exo-Cy7 (low dose) | | |
| 5M | 3 | Exo-Cy7 (high dose) | | |

M- Male; Cy7- unprocessed Cy7

TABLE 6
Study Timeline

| | | | | Test Item | | Termination |
|---|---|---|---|---|---|---|
| | | Body | Clinical | administration | Maestro | and |
| Study Day | Procedure | weight | observation | (IV) | Imaging | tissue harvest |
| Acclimation | | ✓ | | | | |
| l | Dosing | ✓ | ✓ | ✓ | 0.5, 2, 4 hours | |
| | | | Close follow | | after dosing | |
| | | | up during the | | | |
| | | | first two | | | |
| | | | hours and | | | |
| | | | then every | | | |
| | | | two hours | | | |
| | | | until the end | | | |
| | | | of the | | | |
| | | | working day | | | |
| 2 | Termination | ✓ | | | ✓ | ✓ |
| | | | | | | 24 hours |
| | | | | | | post-dosing |

Methods:

Nine mice were used in the study and divided into five groups of one animal per group for control (vehicle and low and high dose of Cy7 fluorophore) and three animals in each group that received Exo-Cy7 at high and low doses Animals received 200 μL of the tested compounds according to study group and fluorescence was measured using Maestro scanner at 0.5, 2, 4 and 24 hours after Test Item administration. At termination liver, lungs, kidneys and urinary bladder were excised and scanned separately. Afterwards, the organs were fixed in 4% Formaldehyde for histopathological analysis.

Results:

Morbidity and Mortality—No animal died during the study.

Body Weight (BW)—Body weight was comparable in all study groups.

Clinical observations—No abnormal findings were observed after Test Items' administration

Gross pathology—No abnormal findings were observed during necropsy.

Biodistribution—The signal was mostly observed in the liver and the urinary bladder. At 30 minutes after administration the fluorescent signal was observed in the liver in groups 4M and 5M (Exo-Cy7 low and high dose, respectively), in the urinary bladder in animals from groups 2M-5M and in the urethra of animals from groups 4M and 5M. The signals in groups 2M-4M (Cy7 low dose, Cy7 high dose and Exo-Cy7, respectively) were comparable, whereas in group 5M (Exo-Cy7 high dose) the signal was significantly higher (p<0.001 4M vs. 5M, liver, 30 minutes, urethra, 30 minutes, urinary bladder, 30 minutes and 2 hrs and p<0.01 4M vs. 5M, 4 hrs, urinary bladder). At 2 hours after administration the fluorescent signal was observed in the liver of animals from groups 2M-4M (Cy7 low and high dose and Exo-Cy7 low dose, respectively), and in the urinary bladder in animals in groups 2M-5M. The highest signal in the urinary bladder was recorded in group 5M (Exo-Cy7 high dose). Lack of signal in the liver of animals from Group 5M might be a result of a technical error, since fluorescent signal was recorded at 4 hours post administration. At 4 hours after administration the signal was recorded in the liver of animals from groups 4M and 5M (Exo-Cy7 low dose and high dose, respectively) and in the urinary bladder in animals from groups 2M-5M. The highest signal in the urinary bladder was recorded in group 5M (ExoCy7 high dose), whereas in the liver the signal in Group 4M and 5M was comparable (slightly higher in Group 4M than in Group 5M). At 24 hours after administration a low signal in the liver was observed only in mice from group 5M (Exo-Cy7 high dose) and in the urinary bladder of animal from groups 2M, 3M and 5M (Cy7 low dose, Cy7 high dose and Exo-Cy7 high dose, respectively). As presented in table 7, at 24 hours after administration fluorescent signal on isolated organs was observed only in organs collected from animals in groups 4M and 5M (Exo-Cy7 low dose and high dose, respectively). The signal was recorded in lungs and liver of animals from both groups and in both kidneys of animals from Group 5M.

TABLE 7
Fluorescent intensity in isolated organs 24 hours after administration.

| Group | | Liver | Lungs | Kidney 1 | Kidney 2 |
|---|---|---|---|---|---|
| 4M (Exo-Cy7—Low dose) | SEM | 3.0 | 20.1 | 0.0 | 0 |
| | Mean | 3.0 | 10.1 | 0.0 | 0 |
| 5M (Ex-Cy7—High dose) | Mean | 19.3 | 21.8 | 5.9 | 5.7 |
| | SEM | 14.1 | 9.7 | 54. | 4.6 |

Conclusion:

In conclusion, exosomes were found in a dose-dependent manner in the liver, the kidneys, the urinary bladder and the lungs, and were retained for up to 24 hours. While their targeting into the liver was clearly exhibited, their appearance in the kidneys and the urinary bladder presented probably their elimination through the urinary track. Their observation in the lungs 24 hours after administration could possibly be due to larger aggregates being trapped in the lungs' bronchioles.

### Example 15

### Experimental Extra-Corporeal Excellasystem Depletion-Replacement on Animal Wistar Rat Model

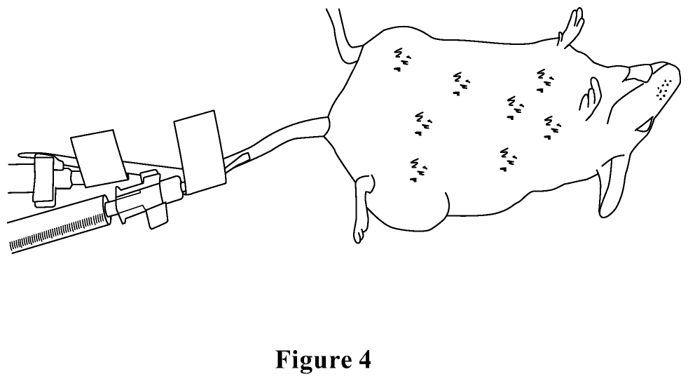

To test feasibility of parallel loop of EV depletion/replacement rat tail model is used where there is an opportunity to collect blood from the artery on the ventral aspect of the rat's tail and delivery (replacement) of rat plasma fused with immunomodulating EVs purchased human cord blood frozen exosomes (EXCBS-F-100, ZenBio) through tail vein delivery (as illustrated in FIG. 4). The ventral caudal artery is used for the collection of small amounts of blood for the depletion procedure (not more than 10-15% of blood). 25G needle is slowly inserted parallel to the tail, just under the skin. For EV enrichment delivered through tail vein is performed as per special operating procedure adopted from UBC Animal Care Guidelines, October 2012 (incorporated herein by reference). Umbilical cord derived EVs are immunosuppressing and selected to be used in immunomodulation in lupus nephritis-LN animal model. BTK inhibition ameliorates kidney disease in spontaneous lupus nephritis.

Alternatively, placental mesenchymal stem cells EVs may be used for immunoprotection in LN animal model. For antiageing effect, stimulated platelets EVs are delivered into rat models provided for ageing research. For obtaining placental derived MSC EVs the following procedure is performed: 3 liters of conditioned medium are collected from placental derived mesenchymal stem cells. Cells are precleaned from debri at 3000 g. The downstream purification of EVs by TFF cross filtration (at 700 Kda filters), followed by 3-5 times washing with 1 liter PBS. Concentrated retanate at 150 ml of TFF filtrated EV enriched isolated fraction and further purify by filter was filtered by 0.5 mcn and prepared for fusion into sterile injector.

In additional experiment to test dialysis patients EV depletion/replacement adjunctive to RBC transfusion , the fusion device was selected comprising a ready to use obtained from public blood bank for transfusion red blood cells-RBC bag packed for transfusion in single used standard package with label. Before transferring into the patient, the RBC bag is injected in an aseptic close loop way (preferably through cannulation) with selected protective healthy donor collected EVS (from PBMC as described below) and is placed for 20 minutes to shake gently.

### Example 16

### Fusion Source

Using plasma derived EVs of healthy donors is an alternative source for EV fusion replacement source and can be applied as a supportive treatment for different pathologies (e.g. as an adjunctive treatment for CKD/end stage renal disease-ESRD in (hemo)dialysis treated patients.), or aging related disorders. Experimental set up standard lyophilized exosomes from healthy donors are purchased from HansaBioMed Life Sciences Ltd. (Lonza).

Allogenic centralized source of GMP supply of EVs from selected source may be stored frozen after separation/purification process. Experimental purification systems tested result in 70-90% of purity by using the following purification tools: classical crossflow devices, With >500 kDa cutoff for EV removal ACTA (GE), PALL-ultrafiltration (Sartorius-VivaFlow), mPES MidiKros, SpectrumLabs, IZON. All are validated for close loop GMP production.

Depletion of pathogenic patient Evs during dialysis outlined as follows. At each hemodialysis session <10% of estimated blood volume (not more than 250 ml) is collected for EV depletion and fusion with donor EVs. Optional use of Aethlon ADAPT™ (adaptive dialysis-like affinity platform technology) system Hemopurifier®, reduce the systemic load of cancer exosomes and can be used as additional loop in hemodialysis machine adapted from apheresis loop description as referenced in Annette M Marleau 1, Chien-Shing Chen, James A Joyce, Richard H Tullis 2012 Exosome Removal as a Therapeutic Adjuvant in Cancer June 27; 10:134. doi: 10.1186/1479-5876-10-134(incorporated herein by reference).

A single unit ultrafiltration device is inserted into the dialysis machine as additional loop with cut off not only for toxins, but also to deplete pathogenically induced already released EV from dialysate and then EV depleted blood cells will enter Replacement EV Fusion Unit. For clinical trial FIH safety the multiple unit system is used. Blood preferably enters EV depletion TFF loop post hemodialysis loop before AV shunt entrance. Collected blood flows through EV depletion module. EV is depleted from patient blood by ultrafiltration using a 700-1000 kDa, then the blood flows to replacement fusion unit. Hemodialyzed blood discarded from ultrafiltrate of dialysate toxins is removed from the patient and flow to Fusion Unit where is co-incubated with EVs supplied (separated/fractionated) from donor platelets for 30 min in 37° C. at slightly basic pH=7.4 balanced by approved systemic buffer solution in order to induce fusion. Then is returned to the patient through the hemodialysis loop.

Preferable fusion system is single use transfusion where 500 ml bag is co-infused with Replacement EVs to enrich 10% of patient blood cells (˜250 ml) depleted from pathogenic EVs—place for 30 min to 2 hrs at 37° C. The blood from the patient is perfused through a conventional hemofilter, which separates the blood into an ultrafiltrate component and a blood cellular concentrate. Then the latter is connected through close loop system into enrichment/replacement unit and is returned to the patient.

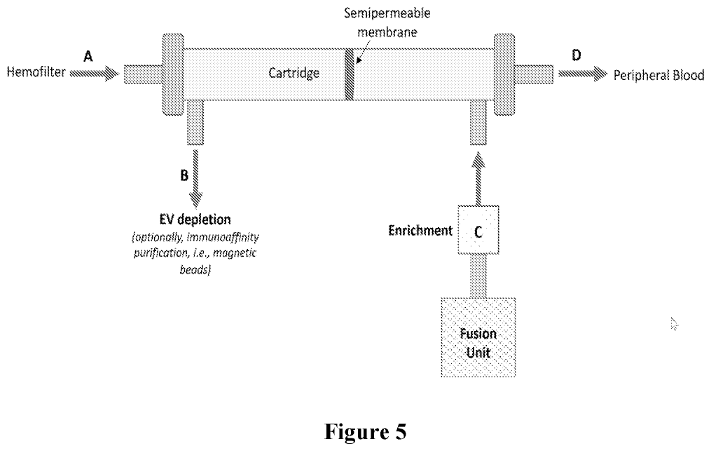

As can be seen in FIG. 5, The ultrafiltrate (optionally hemofiltrate) produced by the hemofilter enters the Excellasystem EV replacement device lumen (A inlet) upon which the pathogenic Evs have been filtrated, or purified by immunoaffinity, and then discarded/depleted (B); The blood from the hemofilter enters the extracapillary space of the hollow fiber cartridge, the blood is separated from the EVs by the semipermeable hollow fiber membrane and enriched with regenerative rEVs (inlet C) post-fused, or fused during perfusion, and the transfused into the patient blood flow—systemic circulation (D outlet). In a case of ESRD patients EV replacement during hemodialysis, blood transfused/dialyzed two perfusion loops. The first one is a conventional hemofiltration loop, the second is the Excelllasystem EV replacement loop.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof. As used herein the terms “comprises”, “comprising”, “includes”, “including”, “having” and their conjugates mean “including but not limited to”. The term “consisting of” means “including and limited to”.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Whenever the term “about” is used, it is meant to refer to a measurable value such as an amount, a temporal duration, and the like, and is meant to encompass variations of ±20%, ±10%, ±5%, ±1%, or ±0.1% from the specified value, as such variations are appropriate to perform the disclosed methods.

It will be understood that when an element is referred to as being “on,” “attached” to, “operatively coupled” to, “operatively linked” to, “operatively engaged” with, “connected” to, “coupled” with, “contacting,” etc., another element, it can be directly on, attached to, connected to, operatively coupled to, operatively engaged with, coupled with and/or contacting the other element or intervening elements can also be present. In contrast, when an element is referred to as being “directly contacting” another element, there are no intervening elements present. Whenever the term “about” is used, it is meant to refer to a measurable value such as an amount, a temporal duration, and the like, and is meant to encompass variations of ±20%, ±10%, ±5%, ±1%, or ±0.1% from the specified value, as such variations are appropriate to perform the disclosed methods.

Whenever terms “plurality” and “a plurality” are used it is meant to include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

By “patient” or “subject” is meant to include any mammal A “mammal,” as used herein, refers to any animal classified as a mammal, including but not limited to, humans, experimental animals including monkeys, rats, mice, and guinea pigs, domestic and farm animals, and zoo, sports, or pet animals, such as dogs, horses, cats, cows, and the like.

“Treating” or “treatment” of a disease as used herein includes: preventing the disease, i.e. causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms, or relieving the disease, i.e., causing regression of the disease or its clinical symptoms.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A process for acellular therapy in a subject in need of such therapy, the process comprising: a) obtaining a biological material from the subject; b) optionally, eliminating vesicle content from the biological material; c) providing a sample of essentially purified therapeutic extracellular vesicles; d) fusing the therapeutic extracellular vesicles of step c with the biological material of step a to obtain a biological material comprising therapeutic extracellular vesicles; and, e) transfusing the biological material of step d to the subject, wherein the transfusion is extracorporeal transfusion.

2. The process of claim 1, wherein the biological material is selected from the group consisting of apheresis or hemodialysis accepted vascular system derived fractions, cells, fluids, blood cells fractions, plasma, body fluids, lymphatic system derived cells, adipose stem cells, stromal vascular fraction (SVF) of adipose tissue , bone marrow fractions, including blood cells, platelets , mesenchymal stem cells, hematopoietic stem cells, soft tissue derived cells, fibroblast-like cells, mesenchymal cells, synovial fluid fractions from joint aspirates (arthrocentesis), peritoneal dialysis accepted fluids, or a combination thereof.

3. (canceled)

4. (canceled)

5. The process of claim 1, wherein the therapeutic extracellular vesicles in the sample are selected from the group consisting of lipid membrane-associated extracellular vesicles, artificial vesicles, pre-manufactured EV mimetics, exosomes, excellamers, acellular vesicles, micro-vesicles, membrane vesicles, apoptotic bodies, autophagosomes, cell fragments-derived membrane-containing particles, nano-ghosts, exomers, lipid membrane nanoparticles, and lipid membrane submicron particles, and organelles-containing membrane-associated particles.

6. (canceled)

7. The process of claim 1, wherein the particle size of the therapeutic extracellular vesicles is between 30 nm to 3000 nm.

8. The process of claim 7, wherein the particle size of the therapeutic extracellular vesicles is between 100 nm to 1000 nm.

9. The process of claim 1, wherein the step of eliminating vesicle content from the biological material is performed by dialysis defiltration, affinity column chromatography, immunoadsorbtion, centrifugation, ultra-centrifugation, cross flow filtration, tangential flow filtration, and ultrafiltration.

10. The process of claim 1, wherein the step of transfusing the biological material of step d to the subject is performed by a recipient-adjusted extracorporeal device.

11. The process of claim 10, wherein the recipient-adjusted extracorporeal device is selected from the group consisting of hollowfiber dialysis device, apheresis, blood transfusion device, plasma transfusion device, blood cells transfusion device, cardiopulmonary resuscitation, extracorporeal membrane oxygenation, hemofiltration, cardiopulmonary bypass, arthrocentesis, peritoneal dialysis, intrathecal device, and a syringe pump.

12. (canceled)

13. (canceled)

14. (canceled)

15. The process of claim 1, wherein the fusion step and the transfusion step are carried out in a closed-loop system.

16. The process of claim 1, wherein the sample of therapeutic extracellular vesicles is prepared immediately prior to the transfusion step, or in advance of the transfusion step.

17. (canceled)

18. The process of claim 17, wherein the sample of therapeutic extracellular vesicle is cryopreserved.

19. (canceled)

20. (canceled)

21. The process of claim 1, wherein the therapeutic extracellular vesicles in the sample carry externally preloaded active therapeutic biomolecule.

22. The process of claim 21, wherein the biomolecule is selected from the group consisting of a small molecule, chemical organic moiety, hormone, neurotransmitter, cytokine, prostaglandin, lipid bioactive moiety, oligonucleotide, dipeptide, oligomer, peptide, protein, antibody, vaccine, DNA, viral vector, RNA, antioxidant, phytotherapeutic material, photosensitive material, electromagnetic-sensitive material, quantum dot, oxygen, and NO donor.

23. The process of claim 1, wherein the biological material comprising the therapeutic extracellular vesicles of step d is stored prior to the transfusion step.

24. The process of claim 23, wherein the biological material comprising the therapeutic extracellular vesicles of step d is cryopreserved.

25. The process of claim 1, wherein the subject in need is afflicted with a condition selected from the group consisting of metabolic disease, kidney disease, liver disease, diabetes, obesity, NASH, cancer, inflammatory disorder, autoimmune disorder, cardiovascular disorder, post-trauma disorder, orthopedic disorder, burns, wound-related hormonal pathology, genetic disorder, neurological disorder, aging-related condition, post-surgery, post-accident stress-related condition, acute radiation syndrome, infection-related condition, condition related to pharma-toxicity, psychiatric disorder, environmental hazards-related toxicity, organ transplantation-related condition, chronic pain, acute pain, neurodegenerative disease, preterm birth-related condition, preeclampsia-related condition, infertility, lung pulmonary respiratory disorder, ischemia, stroke, impotence, skin disorders, condition related to plastic surgery or aesthetic procedures, and blindness.

26. The process of claim 25, wherein said kidney disease comprises End Stage Renal Disease (ESRD).

27. The process of claim 25, wherein said condition comprises aging-related condition and wherein said acellular therapy comprises plasmapheresis of age-pathogenic EVs and fusion of young healthy platelet derived EVs to patient depleted platelets.

28. A system for acellular therapy comprising a)a fusion unit comprising a housing and, optionally, movement-inducing element, said fusion unit is configured to promote fusion of therapeutic extracellular vesicles with a biological material obtained from a human subject to thereby obtain a biological sample comprising said therapeutic extracellular vesicles; b) therapeutic extracellular vesicles supply unit configured to supply a sample of therapeutic extracellular vesicles to the fusion unit; and c)a transfusion unit configured to transfuse the biological material comprising the therapeutic extracellular vesicles to the human subject.

29. The system of claim 28, wherein the fusion unit further comprises a temperature controlling element.

30. The system of claim 28, wherein the fusion unit and the transfusion unit are directly connected with each other, indirectly connected with each other or independent of each other.

31. (canceled)

32. (canceled)
